(19) United States
(12) Patent Application Publication
(10) Pub. No.: US 2023/0233106 A1
(43) Pub. Date: Jul. 27, 2023

(54) Solution for Determination of Supraphysiological Body Joint Movements

(71) Applicant: Kneedly AB, Göteborg (SE)

(72) Inventors: Martin Fagerström, Kållered (SE); Alice Nilsson, Göteborg (SE); Joel Nilsson, Göteborg (SE); Alexander Rydevald, Göteborg (SE); Kristian Samuelsson, Västra Frölunda (SE); Eric Hamrin Senorski, Västra Frölunda (SE); Linn Söderholm, Göteborg (SE)

(21) Appl. No.: 18/011,011

(22) PCT Filed: Jun. 17, 2021
(86) PCT No.: PCT/SE2021/050594

§ 371 Date: Dec. 16, 2022

(30) Foreign Application Priority Data
Jun. 30, 2020 (SE) 2050794-3

(51) Int. Cl. A61B 5/11; G06V 10/75; G06V 10/22; G06T 7/00; G06V 10/25; G06T 7/246

(52) U.S. Cl. CPC A61B 5/1128; G06V 10/755; G06V 10/23; G06T 7/0012; G06V 10/25; G06T 7/248; G06V 2201/033

(57) ABSTRACT

A solution for non-invasive determination of supraphysiological body joint kinematics. The solution obtains external images related to a test procedure of the body joint and performs image analysis on the obtained images to define a pattern of a plurality of spatial points in a region of interest. Each individual spatial point is defined by a unique pattern of neighboring surrounding pixels in each image, and the pattern is part of a high-contrast speckle pattern applied to the body joint. The solution identifies displacements of the spatial points in subsequently obtained images by tracing a location of the unique pattern of neighboring pixels in each image in relation to a base image of the body joint, calculates deformation measures from the displacements of the plurality of spatial points, and obtains deformation measures of a reference body joint. The solution compares the deformation measures and determines supraphysiological body joint kinematics from the comparison.

### TECHNICAL FIELD

The present invention relates to a system, method and arrangements for determination of supraphysiological body joint movements and in particular a solution for non-invasive examination using digital image analysis.

### BACKGROUND

There are a number of different methods for observing supraphysiological joint kinematics, for instance for determining abnormal movements of a body joint, used in the clinical area. Most of these rely on an examiner, e.g. a physician or other medical personnel, performing a medical test on a test subject being e.g. a human or an animal. Understanding the movements of body joints can prove to be difficult, especially with regard to the interpretation of the movements and associating these to specific problems or injuries of the joint; furthermore, the interpretation can vary depending on the examining person, i.e. inter-rater variability.

Currently, there are several different diagnostic methods used depending on the type of joint and type of injury. For instance, for the cruciform ligament of the knee joint's related abnormal movement, the methods are divided into methods for examining the Anterior Cruciate Ligament (ACL) or the Posterior Cruciate Ligament (PCL). In case of the ACL, some of the manual tests are Pivot shift test, Lachman's test, Lelli's tests, and anterior drawer test. For PCL, the posterior drawer test or ocular observation of posterior sag signs may be used.

For studies of collateral ligaments of the knee joint, Valgus or Varus stress tests are used for medial collateral ligament and lateral collateral ligament, respectively.

Supporting technologies for the different methods for examining kinematics of body joints and/or ligaments may include internal or external studies, i.e. inside the body or observing behavior outside the body, respectively. The internal studies may for instance comprise microwave tomography, magnetic resonance imaging (MRI), ultra sound, or dynamic stereo x-ray imaging (DSX) to understand the movements and deformations of the ligament as well as the behavior of the joint. The different techniques for internal studies of for instance the knee, has limited validity and reliability or require large amount of resources and costly equipment. Furthermore, imaging techniques for external studies of the ligaments exists but have low reliability.

There is thus a need for cost effective and reliable solutions for supporting studying supraphysiological joint kinematics to support the diagnosis of abnormal movement of body joints/ligaments.

### SUMMARY

It is an object to obviate at least some of the above disadvantages and provide improved systems, method, and computer-readable storage medium for determination of supraphysiological body joint movements and a solution for non-invasive examination using digital image analysis, for instance for indicating internal injuries.

This is provided in a number of embodiments, in which a first is a system for non-invasive determination of supraphysiological body joint kinematics. The system may identify and determine supraphysiological body joint kinematics for human, animals or any other suitable means. The system comprising at least one digital camera, an electronic device with at least one processing unit, at least one computer-readable memory, at least one user interface, and at least one camera interface. The processing unit is arranged to execute instruction sets stored in the computer-readable memory to obtain images related to a test procedure of the body joint from the at least one digital camera connected to the communication port, performing image analysis on the obtained images to define a pattern of a plurality of spatial points in a region of interest; wherein each individual spatial point is defined by a unique pattern of neighboring surrounding pixels in each image, and where the pattern is part of a high-contrast speckle pattern applied to the body joint, identify displacements of the spatial points in subsequently obtained images by tracing a location of the unique pattern of neighboring pixels in each image in relation to a base image of the body join, from the displacements of the defined plurality of spatial points calculating deformation measures, obtain deformation measures of a reference body joint, compare deformation measures between the body joint and the reference body joint, and determine supraphysiological body joint kinematics from the comparison.

Furthermore, the processing unit may be arranged to operate a machine learning function or artificial intelligence function to build a database with diagnoses of damaged body joints and selecting a diagnosis from the database for a specific supraphysiological movement.

The strains may be calculated as a function of a gradient of a displacement field by calculating the Green-Lagrange strain, which is a representative measure of a deformation of an analyzed surface.

Another aspect of the present invention is provided, a method, in an electronic device, for determining supraphysiological body joint kinematics. The method comprising steps of measuring deformations of an applied high contrast speckle pattern on a body joint, wherein measuring deformations comprises obtaining, directly or indirectly, from at least one camera, images of the applied high contrast speckle pattern on the body joint related to a test procedure of the body joint, performing image analysis of the obtained images to define a plurality of spatial points by identifying a unique neighboring pattern of surrounding pixels in the images, identifying a displacement and its potential variation over time of a same spatial points in subsequent images by using the neighboring pixels, and from the displacements of the defined plurality of spatial points calculating deformation measures. The method further comprises obtaining deformation measures for a reference body joint, comparing deformation measures between the body joint and the reference body joint, and determining supraphysiological body joint kinematics from the comparison.

The measuring may further comprise an artificial intelligence function, comparing said movements and/or said strains, respectively, to reference movement values and to reference strain values, respectively, and identifying normal and/or abnormal movements and/or normal and/or abnormal strains, respectively.

In the system and method, the image analysis may be performed using digital image correlation, DIC, analysis.

The method may be arranged to analyze at least one of a knee joint, elbow joint, hip joint, ankle joint, or shoulder joint.

Yet another aspect of the present invention is provided, an electronic device comprising: at least one processing unit, at least one computer-readable memory, at least one user interface, and at least one communication port, wherein the at least one processing unit is arranged to execute one or more programs including instructions for performing the method of any of the embodiments herein.

Still another aspect of the present invention is provided, a computer-readable storage medium storing one or more programs configured to be executed by one or more processors of an electronic device, the one or more programs including instructions for performing the method of any of the embodiments herein.

The proposed solution makes it possible to achieve an efficient and cost-effective solution for non-invasive determination of supraphysiological body joint kinematics.

This has the advantages of providing a cost-efficient solution for determining and diagnosing abnormal movements of body joints and ligaments. As being based on image analysis and post-processing of strains, the proposed solution can be easily made portable for usage with handheld devices such as smartphones and tablets. The proposed solution also provides a non-invasive method to objectively assess the presence of joint injury, without being dependent on the skills of the person performing the medical examination.

According to another aspect of the present disclosure, there is provided a system for non-invasive determination of supraphysiological body joint kinematics. The system comprising at least one digital camera, an electronic device with at least one processing unit, at least one computer-readable memory, at least one user interface, and at least one camera interface. The processing unit is arranged to execute instruction sets stored in the computer-readable memory to obtain images related to a test procedure of the body joint from the at least one digital camera connected to the communication port. Further, performing image analysis on the obtained images to define a pattern of a plurality of spatial points in a region of interest; wherein each individual spatial point is defined by a unique pattern of neighboring surrounding pixels in each image, and where the pattern is part of a high-contrast speckle pattern applied to the body joint. Moreover, identify displacements of the spatial points in subsequently obtained images by tracing a location of the unique pattern of neighboring pixels in each image in relation to a base image of the body joint. Furthermore, from the displacements of the defined plurality of spatial points calculating deformation measures determine supraphysiological body joint kinematics.

The processing unit in accordance with the other aspect of the system may be further arranged to operate a machine learning function or artificial intelligence function to build a database with diagnoses of damaged body joints and selecting a diagnosis from the database for a specific supraphysiological movement.

According to another aspect of the present disclosure, there is provided a method in (performed by) an electronic device, for determining supraphysiological body joint kinematics, comprising steps of, measuring deformations of an applied high contrast speckle pattern on a body joint, wherein measuring deformations comprises: obtaining, directly or indirectly, from at least one camera, images of the applied high contrast speckle pattern on the body joint related to a test procedure of the body joint; performing image analysis of the obtained images to define a plurality of spatial points by identifying a unique neighboring pattern of surrounding pixels in the images; identifying a displacement and its potential variation over time of same spatial points in subsequent images by using the neighboring pixels; and from the displacements of the defined plurality of spatial points calculating deformation measures.

Further, the method comprises the step of determining supraphysiological body joint kinematics.

### BRIEF DESCRIPTION OF THE DRAWINGS

In the following section, the invention will be described in a non-limiting way and in more detail with reference to exemplary embodiments illustrated in the enclosed drawings, in which:

FIG. 1 is a schematic block diagram illustrating an example system according to the present invention;

FIGS. 2 is a schematic block diagram illustrating an exemplary device according to the present invention;

FIG. 3 is a schematic block diagram illustrating an exemplary method according to the present invention;

FIG. 4 is a schematic block diagram illustrating an exemplary user interface; and

FIG. 5 is a schematic block diagram illustrating an exemplary detailed method according to the present invention.

### DETAILED DESCRIPTION

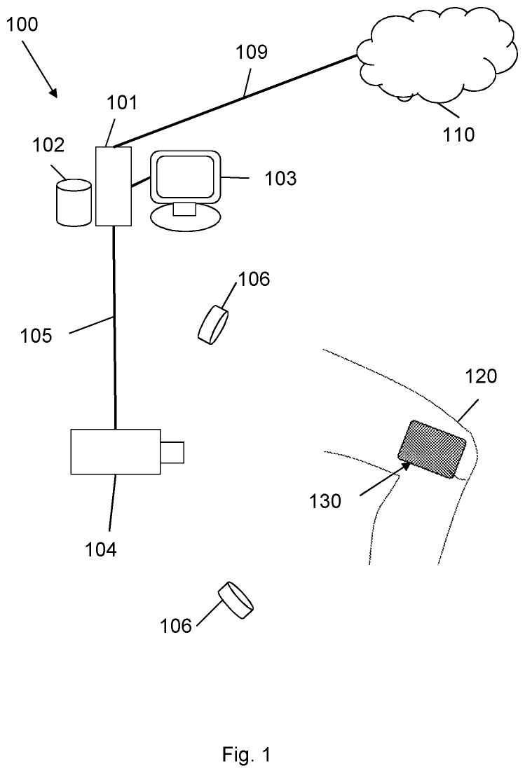

In FIG. 1 reference numeral 100 generally denotes a system for determining and identifying supraphysiological kinematics of human body joints 120 and optionally diagnosing supraphysiological or abnormal joint kinematics of the body joint. The system comprises an electronic processing device 101 optionally connected to a database 102 with stored data for supporting determining and diagnosing the supraphysiological movements. Furthermore, the system comprises one or more user interfaces, e.g. a display 103 with or without touchscreen functionality, keyboard (not shown), mouse (not shown), digital pen (not shown), and so on. The system may also comprise a camera 104, for obtaining digital images of the body joint to be analyzed, connected 105 to or integrated with the electronic processing device. One or several light sources 106 may also be provided to facilitate a uniform lighting condition. Furthermore, the processing device may be connected to an internal or external network 110 using a communication link 109. Even though FIG. 1 generally illustrates a system for determining and identifying supraphysiological kinematics of human body joints, the system may in accordance with the present disclosure determine and identify supraphysiological kinematics for animal body joints also.

During operation of the system, the body joint 120 to be analyzed is provided with a pattern 130, e.g. a randomized pattern or a regular or semi-regular pattern. In one example, the pattern is provided using a sponge with ink or some other paint-like substance that adheres to the skin of the body joint and provides a random pattern. In another example, the pattern is provided on a thin sheet of flexible material adhered to the skin.

According to another aspect of the disclosure, FIG. 1 provides a system 100 for non-invasive determination of supraphysiological body joint 120 kinematics. The system 100 comprising at least one digital camera 104, an electronic device 101 with at least one processing unit 201, at least one computer-readable memory 202, at least one user interface 103, and at least one camera interface 210, wherein the processing unit is arranged to execute instruction sets stored in the computer-readable memory to obtain images related to a test procedure of the body joint from the at least one digital camera connected to the communication port. Further, its arranged to perform image analysis on the obtained images to define a pattern of a plurality of spatial points in a region of interest, wherein each individual spatial point is defined by a unique pattern of neighboring surrounding pixels in each image, and where the pattern is part of a high-contrast speckle pattern 130 applied to the body joint 120. Further, its arranged to identify displacements of the spatial points in subsequently obtained images by tracing a location of the unique pattern of neighboring pixels in each image in relation to a base image of the body joint. Moreover, from the displacements of the defined plurality of spatial points calculating deformation measures. The unit is arranged to determine supraphysiological body joint kinematics.

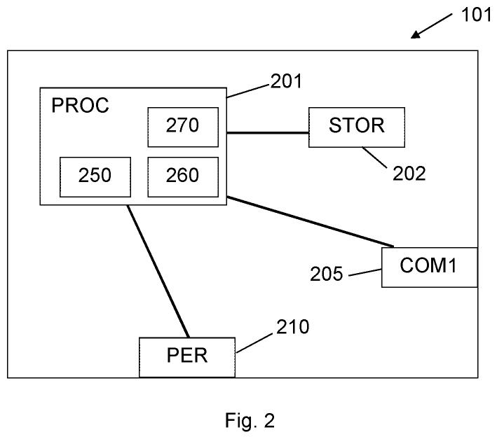

FIG. 2 is a block diagram illustrating the electronic processing device 101. Device 101 includes at least one memory 202 (which optionally includes one or more computer-readable storage mediums), memory controller (not shown), one or more processing units 201, one or more interfaces 210 for peripherals or other input control devices internal or external the electronic processing device, for instance one or more external cameras may be connected via this interface. However, in case of a system with a built-in camera, an internal camera interface may be used. These components optionally communicate over one or more communication buses or signal lines. The processing units may for instance comprise a central processing unit (CPU), a graphical processing unit (GPU), a digital signal processor (DSP), an application specific integrated circuit (ASIC), a field programmable gate array (FPGA), or a combination of these or any other suitable digital computational device.

Memory 202 of electronic device 101 can include one or more computer-readable storage mediums, for storing computer-executable instructions, which, when executed by one or more computer processors 201, for example, can cause the computer processors to perform the techniques described below. A computer-readable storage medium can be any medium that can tangibly contain or store computer-executable instructions for use by or in connection with an instruction execution system, apparatus, or device. In some examples, the storage medium is a transitory computer-readable storage medium. In some examples, the storage medium is a non-transitory computer-readable storage medium. The computer-readable storage medium can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like.

Furthermore, the electronic device may comprise a communication interface 205 for transmitting and receiving signals to other computational devices such as remotely located servers and the like using a wired or wireless connection 109. The communication interface may for instance be connected to a network 110 such as an intranet or extranet, for instance Internet. The connection 109 may for instance be arranged to operate according to the Ethernet protocol or other similar digital data protocols.

The processing unit 201 may comprise a plurality of functional modules, 250, 260, 270 for operating and controlling different functionality of the processing unit as will be discussed in more detail below. For instance, the processing unit may comprise a computational and analysis module 250 operating instruction sets for performing different functionality such as image analysis and so on, a user interface module 260 handling incoming user commands and outgoing user information, and a peripheral and communication control module 270 for handling user interface commands and controlling communication signals to/from peripheral devices and external computational devices.

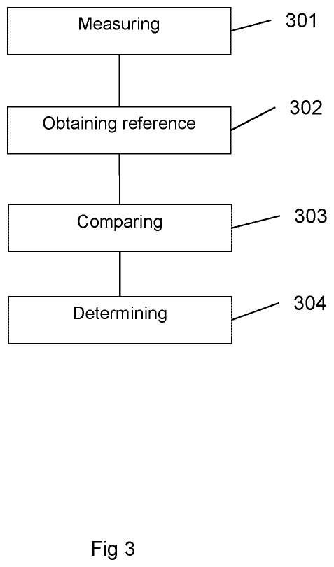

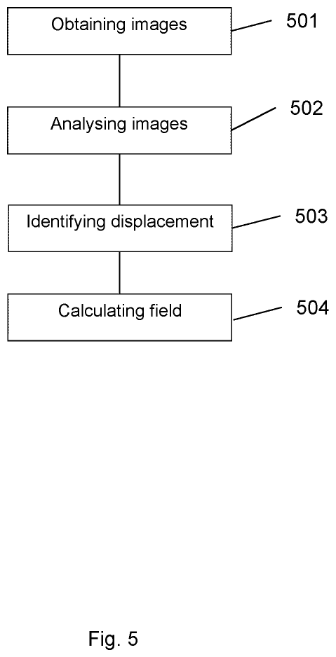

With reference to FIGS. 3 and 5, a method for determining and identifying joint kinematics, for instance supraphysiological movements of body joints and optionally diagnosing supraphysiological movements will now be described. The system uses one or more cameras (for example one or two cameras), for obtaining 501 a series of images of the body joint to be analyzed. The system and method e.g. comprises a Digital Image Correlation (DIC) solution, and measures 301 relative movements outside the joint of interest, e.g. on body skin outside the joint, during tests of the joint. In one embodiment the body joint is a knee joint of a human test subject but it should be noted that measurements of other joints might be of interest and find applicability of the solution as described herein, such as elbow joint, hip joint, ankle joint, shoulder joint, and so on.

The skin of the body joint is prepared with a pattern, e.g. a black and white pattern or similar high contrast pattern, and the DIC process determines movement of the pattern relative an initial base sample of the body joint. Analyzed movement of the pattern may be compared to reference samples to determine supraphysiological movement, for instance from a similar joint of the test subject or a reference joint stored in a database. It should be noted that the reference data may also comprise analyzed deformation measures indicative of supraphysiological movement.

The method may be used to identify, determine and optionally diagnose supraphysiological joint movements, for instance in injured joints, e.g. injured knees:

A supraphysiological movements can appear, in injured knees, where increased anteroposterior and rotational movements/laxity will occur if any of the cruciform ligaments are injured. The cruciform ligaments are e.g. Anterior Cruciate Ligament (ACL) and Posterior Cruciate Ligament (PCL).
Collateral ligaments prevent knee joint from bending side-ways and prevents the femur and tibia to separate side-ways. Here the supraphysiological movements can appear in the way that the tibia is pulled side-ways in relation to the femur if the ligaments are injured.
Tests for Cruciate ligaments include, but are not limited to: Drawer test and Lachman's test
Tests for Collateral ligaments in knees include, but are not limited to: Valgus Stress Test and Varus stress test

A suitable pattern, for instance a high contrast speckle pattern, such as a black and white speckle pattern, is applied on the body joint of interest on a test subject. This pattern may be a randomized pattern applied to the skin of the test subject. In one embodiment, the pattern is applied by optionally first painting the joint area white and thereafter sprayed with black spray paint to give a unique pattern, for instance a speckle pattern. Alternatively, the pattern (in black, white or any other suitable color) is provided by providing a sponge or similar soft material with ink or a paint-like substance and then pressing the sponge lightly onto the skin at different locations leaving a pattern of ink or paint-like substance on the skin. However, it should be noted that the method of application of pattern may differ and any suitable method may be used. Alternatively, the pattern is provided on a flexible sheet that may be glued to the body joint and the sheet flexes and follows the movement of the joint during test of the joint. Furthermore, it should be noted that the pattern may be of other colors than black and white as long as the pattern has a suitable high contrast to facilitate image analysis. Furthermore, the pattern need not be randomized but can be of any suitable pattern for detecting movement and strains during movement of the body joint.

One or several cameras are preferably mounted on a rigid support and optionally calibrated, for instance by setting the distance, aperture, or similar technical settings in order to obtain high quality images during the measurement. However, it should be noted that alternatively the camera may be hand-held during image acquisition. For two-dimensional measurement, one camera is normally sufficient, but for three-dimensional measurements, at least two cameras are preferably used. Using three-dimensional measurements will potentially provide a higher degree of accuracy by compensating for unwanted movement of the body joint towards or away from the camera.

During tests of body joints, the joint to be analyzed is moved by the subject or controlled movement is provided by an examiner, such as a physician, or a test robot. The movement is according to a reference pattern following a medical examination method used, and the one or more cameras will obtain 501 a series of images or a film sequence and transmit these directly or indirectly to the processing device. It should be noted that, alternatively, the camera record images during the test and at the end of the test all images are provided to the processing device or the images are stored in the camera or in a storage location (not shown) for a later analysis by a processing device. A reference image series may be obtained from a reference joint, for instance when analyzing a damaged joint, the other joint of the same subject may be used as reference joint

On all obtained images, a pattern of spatial points related to the applied speckle pattern are defined by software analysis. Image analysis 502 is then used to identify a neighboring pattern of surrounding pixels, e.g. a grey scale pattern. These neighboring pixels are then used to identify 503 each corresponding same point in subsequent images and identify a displacement of the same point in subsequent images as compared to an initial base image of the joint or as compared to each preceding image of the joint. The base image provides a starting point when analyzing and determining the displacement of the spatial points and from these determine deformation. The base image may be a first image of the joint before test starts and the joint is still in a relaxed position or in a suitable reference position for the medical test to be performed.

The relative movement of a same point between two images or between the last obtained image and the base image defines a spatial displacement in space and time. The method calculates 504, from the displacements of the defined plurality of spatial points, relevant deformation measures, and how they vary in space and time, e.g. by calculating a spatially varying displacement field from the displacement of the spatial points. Strains may then be calculated as a function of a gradient of the displacement field related to the displacement in time. The relevant deformation measures are compared 303 with corresponding deformation measures of the reference joint. The comparison may be used to determine 304 supraphysiological body joint (120) kinematics.

In one example, the deformation measures, such as strain gradients, may be used to calculate a Green-Lagrange strain which is a representative measure of the deformation of the analyzed surface, free from effects of rigid body movements. However, it should be noted that the analysis is not restricted to the Green-Lagrange strain measure, but that other strain and/or displacement measures may be utilized as well.

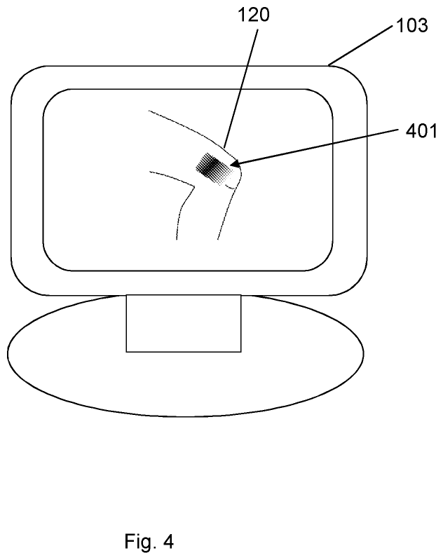

The processing device is arranged to acquire or obtain images related to tests of body joints and analyze the images according to the digital image correlation (DIC) method. Preferably, the processing device is arranged to provide the result of the analysis to a user of the processing device, e.g. control a user monitor to visually show an image of the joint overlaid with an analyzed strain or displacement field image for comparison together with a similar image of the reference body joint. Such an image is shown in FIG. 4, where the strain or displacement field 401 is overlaid on the image of the body joint 120 in the user interface monitor 103. However, the processing device may be arranged to provide some other measure of strain or displacement such as an average strain over a specific area.

An analyzed reference image or images may be obtained 302 for a reference body joint that may be used during the analysis for comparing 303 analyzed data between body joint with supraphysiological (e.g. abnormal) movement and the reference body joint. For instance, when analyzing a damaged joint of a test subject, the other joint of the same subject may be used as reference joint. However, it should be noted that reference data for the body joint behavior may be obtained from stored data in a database. Such stored data may for instance comprise analyzed displacement fields for similar test measurements as for the current test on a similar body joint with known behavior. Furthermore, specific displacement fields may be determined as relating to supraphysiological movement without comparison with reference data (disclosed in accordance with the other aspect of FIG. 1). In some cases the system only provide the displacement field analysis and representative image for the strains or displacement fields and the examiner may determine supraphysiological movement of the joint.

It should be noted, that according to some aspects of the disclosure, the steps of obtaining 302 and comparing 303 are not performed when operating the method 300.

In one embodiment, one camera is used. However, for better precision, one may use two (or more) synchronized cameras. This will give a possibility to measure 3-dimensional movement (for instance also movements towards the camera) and it will make it possible to study displacements on curved objects (like the knee) with higher precision and thus determine strains and/or displacements with higher precision.

The system and method may be arranged to obtain a series of images during movement of the body joint and use this series to analyze together with the base image and with the reference image or reference images of a reference joint. The system and method may further be arranged to provide a final result, such as an analyzed image that shows a representative displacement or strain field for further analysis either by a user and/or a diagnosis function in the system. Alternatively, the system may be arranged to provide a movie sequence illustrating the change of the displacement field during movement of the body joint and provide this movie sequence to the user for further analysis and/or to the diagnosis function for further analysis. This supports the examiner in determining potential supraphysiological movement of the joint and ligaments.

The system may further comprise a diagnosis function, that analyze the result of the image analysis, e.g. the strain or displacement field and compare strain or displacement fields from known supraphysiological or abnormal movements either directly or using a machine learning function or artificial intelligence function. These functions may also be used to analyze and store measurements during operation in order to train diagnosis and improve the accuracy in determining the supraphysiological movement. The stored data may be anonymized in order to more easily provide such data to third parties, for instance as a database connected to a computer program product for determination of supraphysiological body joint kinematics. The measuring may for instance comprise an artificial intelligence function, comparing said movements, displacements, and/or said strains, respectively, to reference movement values and to reference strain values, respectively, and identifying normal and/or abnormal movements and/or normal and/or abnormal strains, respectively.

These machine learning or artificial intelligence functions may use different types of suitable algorithms, such as supervised or unsupervised learning, reinforcement learning, feature learning, association rules learning, or other similar techniques using any suitable model such as decision-tree analysis, neural networks, support vector machines, regression analysis, Bayesian networks, or genetic algorithms.

The above described functionality may be at least in part be provided as a computer-readable storage medium storing one or more programs configured to be executed by one or more processors of an electronic device, the one or more programs including instructions for performing the method as discussed previously. The instructions may be stored in a computer program product that may be distributed via storage medium or via a communication network.

Furthermore, the system may be provided in a combined housing with for instance the electronic device with the processing unit, memory, and so on, the user interface, and optionally with the camera in the same housing as a stand-alone product for operating the method according to the present solution. In such a solution, the user interface is advantageously a touchscreen user interface. For example, smartphones and tablets with a built-in camera may be used as a stand-alone product operating functionality according to the present invention.

It should be noted that the word “comprising” does not exclude the presence of other elements or steps than those listed and the words “a” or “an” preceding an element do not exclude the presence of a plurality of such elements. It should further be noted that any reference signs do not limit the scope of the claims, that the invention may be at least in part implemented by means of both hardware and software, and that several “means” or “units” may be represented by the same item of hardware.

The above mentioned and described embodiments are only given as examples and should not be limiting to the present invention. Other solutions, uses, objectives, and functions within the scope of the invention as claimed in the below described patent embodiments should be apparent for the person skilled in the art.

1. A system for non-invasive determination of supraphysiological body joint kinematics, the system comprising:
at least one digital camera; and
an electronic device with at least one processing unit, at least one computer-readable memory, at least one user interface, and at least one camera interface ;
wherein the processing unit is arranged to execute instruction sets stored in the computer-readable memory to:
obtain images related to a test procedure of the body joint from the at least one digital camera connected to the communication port;
perform image analysis on the obtained images to define a pattern of a plurality of spatial points in a region of interest, wherein each individual spatial point is defined by a unique pattern of neighboring surrounding pixels in each image, and where the pattern is part of a high-contrast speckle pattern applied to the body joint;
identify displacements of the spatial points in subsequently obtained images by tracing a location of the unique pattern of neighboring pixels in each image in relation to a base image of the body joint;
calculate deformation measures from the displacements of the defined plurality of spatial points;
obtain deformation measures of a reference body joint;
compare deformation measures between the body joint and the reference body joint; and
determine supraphysiological body joint kinematics from the comparison.

2. The system according to claim 1, wherein the processing unit is further arranged to operate a machine learning function or artificial intelligence function to build a database with diagnoses of damaged body joints and select a diagnosis from the database for a specific supraphysiological movement.

3. The system according to claim 1, wherein the deformation measures are strains or deformations calculated as a function of a gradient of a displacement field related to the deformation measures by calculating a Green-Lagrange strain, which is a representative measure of a deformation of an analyzed surface.

4. A method, in an electronic device, for determining supraphysiological body joint kinematics, the method comprising the steps of:
measuring deformations of an applied high contrast speckle pattern on a body joint, wherein measuring deformations comprises:
obtaining, directly or indirectly, from at least one camera, images of the applied high contrast speckle pattern on the body joint related to a test procedure of the body joint;
performing image analysis of the obtained images to define a plurality of spatial points by identifying a unique neighbouring pattern of surrounding pixels in the images;
identifying a displacement and its potential variation over time of same spatial points in subsequent images by using the neighbouring pixels; and
calculating deformation measures from the displacements of the defined plurality of spatial points;
obtaining deformation measures for a reference body joint;
comparing deformation measures between the body joint and the reference body joint; and
determining supraphysiological body joint kinematics from the comparison.

5. The method according to claim 4, wherein the measuring further comprises an artificial intelligence function, comparing said movements and/or said strains, respectively, to reference movement values and to reference strain values, respectively, and identifying normal and/or abnormal movements and/or normal and/or abnormal strains, respectively.

6. The method according to claim 4, wherein deformation measures are strains or deformations calculated as a function of a gradient of displacement field performed by calculating a Green-Lagrange strain, which is a representative measure of a deformation of an analyzed surface.

7. The method according to claim 4, wherein the image analysis is performed using digital image correlation, DIC, analysis.

8. The method according to claim 4, wherein the method is arranged to analyze at least one of a knee joint, elbow joint, hip joint, ankle joint, or shoulder joint.

9. An electronic device comprising:
at least one processing unit;
at least one computer-readable memory;
at least one user interface; and
at least one camera interface;
wherein the at least one processing unit is arranged to execute one or more programs including instructions for performing the method of claim 4.

10. A computer-readable storage medium storing one or more programs configured to be executed by one or more processors of an electronic device, the one or more programs including instructions for performing the method of claim 4.

11. A system for non-invasive determination of supraphysiological body joint kinematics, the system comprising:
at least one digital camera;
an electronic device with at least one processing unit, at least one computer-readable memory at least one user interface and at least one camera interface;
wherein the processing unit is arranged to execute instruction sets stored in the computer-readable memory to:
obtain images related to a test procedure of the body joint from the at least one digital camera connected to the communication port;
performing perform image analysis on the obtained images to define a pattern of a plurality of spatial points in a region of interest, wherein each individual spatial point is defined by a unique pattern of neighboring surrounding pixels in each image, and wherein the pattern is part of a high-contrast speckle pattern applied to the body joint;
identify displacements of the spatial points in subsequently obtained images by tracing a location of the unique pattern of neighboring pixels in each image in relation to a base image of the body joint;
calculate deformation measures from the displacements of the defined plurality of spatial points; and
determine supraphysiological body joint kinematics.

12. A method, in an electronic device, for determining supraphysiological body joint kinematics, the method comprising the steps of:
measuring deformations of an applied high contrast speckle pattern on a body joint, wherein measuring deformations comprises:
obtaining , directly or indirectly, from at least one camera, images of the applied high contrast speckle pattern on the body joint related to a test procedure of the body joint;
performing image analysis of the obtained images to define a plurality of spatial points by identifying a unique neighbouring pattern of surrounding pixels in the images;
identifying a displacement and its potential variation over time of same spatial points in subsequent images by using the neighbouring pixels; and
calculating deformation measures from the displacements of the defined plurality of spatial points; and
determining supraphysiological body joint kinematics.

13. An electronic device comprising:
at least one processing unit;
at least one computer-readable memory;
at least one user interface; and
at least one camera interface;
wherein the at least one processing unit is arranged to execute one or more programs including instructions for performing the method of claim 12.

14. A computer-readable storage medium storing one or more programs configured to be executed by one or more processors of an electronic device, the one or more programs including instructions for performing the method of claim 12.
